(19) United States
(12) Patent Application Publication
(10) Pub. No.: US 2023/0024274 A1
(43) Pub. Date: Jan. 26, 2023

(54) DIETARY BUTYRATE

(71) Applicant: SOCIETE DES PRODUITS NESTLE S.A., Vevey (CH)

(72) Inventor: AMAURY PATIN, Ithaca, NY (US)

(21) Appl. No.: 17/756,887

(22) PCT Filed: Dec. 4, 2020
(86) PCT No.: PCT/EP2020/084731

§ 371 Date: Jun. 3, 2022

(30) Foreign Application Priority Data
Dec. 5, 2019 (EP) 19213720.6

(51) Int. Cl. A23L 33/12; A23L 33/00; A23L 29/00

(52) U.S. Cl. CPC A23L 33/12; A23L 33/40; A23L 29/04

(57) ABSTRACT

A nutritional composition for providing a source of butyrate or butyric acid comprising a compound having the formula (1) wherein R⁶ is along chain fatty acid having between 16 and 20 carbons.

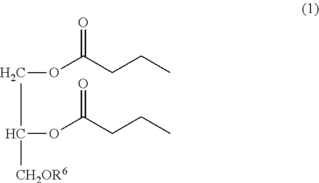

### FIELD OF THE INVENTION

The present invention relates to a dietary source of butyrate.

### BACKGROUND TO THE INVENTION

Salts and esters of butyric acid are known as butyrates or butanoates. Butyric acid in ester form is found in many foods such as milk, especially goat, sheep, cow, camel and buffalo milk, and milk-derived products such as butter as well as cheeses such as parmesan cheese. Butyric acid is also a product of anaerobic fermentation, for example, as a product of fermentation produced by gut microbiota.

The multiple beneficial effects of butyrate are well documented in mammals and livestock. At the intestinal level, butyrate plays a regulatory role on transepithelial fluid transport, mucosal inflammation and oxidative status, reinforces intestinal barrier function, and influences visceral sensitivity and intestinal motility.

Butyrate has been shown to improve the intestinal structure of piglets with short-bowel syndrome (Bartholome et al., J of Parenter Enteral Nutr. 2004; 28(4):210-222) and decrease the proliferation of colon cancer cells in human cell lines (Lupton, J Nutr., 2004; 134(2):479-482). The production of volatile fatty acids such as butyric acid from fermentable fibers may contribute to the role of dietary fiber in colon cancer (Lupton, The Journal of Nutrition. 134 (2): 479-82). Short-chain fatty acids (SCFA), which include but are not restricted to acetic, propionic and butyric acid, are produced by colonic bacteria that feed on, or ferment non-digestible fiber and/or prebiotics. SCFA, and most notably butyrate, promote regulatory T cells in the colon via histone deacetylase inhibition at the Foxp3 locus (Furusawa Y, et al., Nature 2013; 504(7480):446-450). Oral butyrate supplementation promotes antibacterial activity in intestinal macrophages and restricts dissemination of bacteria beyond the intestinal barrier. Butyric acid also benefits the colonocytes by increasing energy production. Additionally, butyrate has been shown to decrease the incidence of diarrhea (Berni Canani et al., Gastroenterol., 2004; 127(2):630-634), improve gastrointestinal symptoms in individuals with diarrhea-predominant irritable bowel syndrome (Scarpellini et al., Dig Liver Dis., 2007; 1(1):19-22) and enhance the development of the small intestine in neonatal piglets (Kotunia et al., J Physiol Pharmacol. 2004; 55(2):59-68).

Tributyrin is a triglyceride made of three ester functional groups with three butyrate moieties and the glycerol backbone. Under hydrolysis conditions such as those occurring during digestion, tributyrin is potentially a source of three moles of butyric acid per mole of tributyrin. However, the efficacy of tributyrin is potentially limited by its rapid gastric lipolysis.

Butyric acid and tributyrin are both food additives that are generally regarded as safe (GRAS) (21CFR582.60 and 21CFR184.1903 respectively), and are natural components of many dairy items. However, butyric acid is associated with negative sensory qualities such as vomit-like, fecal, and cheesy aroma attributes. Tributyrin also has negative sensory qualities, in particular high bitterness. These unpleasant taste and odor attributes can make the oral administration of compositions including these compounds particularly difficult, especially in the pediatric population.

Accordingly, it would be beneficial to provide a food-grade source of butyrate having improved properties as compared to available solutions. A liquid format would provide further benefit, due to ease of formulation and reduced dissolution and homogenization issues.

### SUMMARY OF THE INVENTION

The present invention provides compounds that are an edible source of butyrate. The present invention provides compounds that have improved organoleptic properties. In particular, the compounds have improved odor and/or taste relative to butyric acid, butyrate salts and tributyrin. The compounds may be used as a dietary source of butyric acid. The compounds may be used in, for example, nutritional compositions for human or animal consumption, including dietary supplements, medical foods, foods for special medical purposes, fortified foods, infant formulas and follow-on formulas.

Advantageously compounds of the present invention may provide an effective delivery of butyric acid to the intestinal compartment.

According to a first aspect of the present invention there is provided use of a compound having the formula

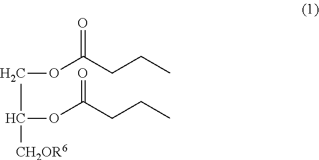

wherein R⁶ is a long chain fatty acid having between 16 and 20 carbons,

in a nutritional composition, for providing a dietary source of butyric acid or butyrate with improved organoleptic properties.

The compound of formula (1) may be present in, for example, a nutritional composition such as a nutritional formulation, a fortified food, a medical food or food for special medical purposes, a dietary supplement, an infant formula, or a follow-on formula.

In one embodiment, the improved organoleptic properties are improved odour. In one embodiment, the improved organoleptic properties are improved taste. In one embodiment, the improved organoleptic properties are improved odour and improved taste. In one embodiment, the improved taste is reduced bitterness.

According to an aspect of the present invention there is provided a nutritional composition for providing a source of butyrate or butyric acid comprising a compound having the formula

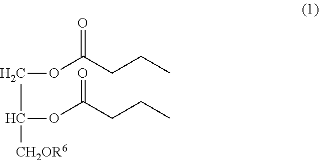

wherein R⁶ is a long chain fatty acid having between 16 and 20 carbons, as the main butyrate moiety containing triglyceride in the nutritional composition.

In a preferred embodiment the compound of formula (1) is the main source of the butyrate or butyric acid, in the nutritional composition.

In an embodiment the compound of formula (1) may provide at least 20%, at least 30%, at least 40%, at least 50%, or at least 60%, at least 70%, at least 80%, or at least 90%, of the butyrate or butyric acid provided by the nutritional composition.

In an embodiment the compound of formula (1) may provide at least 20%, at least 30%, at least 40%, at least 50%, or at least 60%, at least 70%, at least 80% or at least 90%, by weight of the butyrate moiety containing triglycerides in the nutritional composition.

In another aspect the compound of formula (1) provides at least 95% by weight, or substantially all of the butyrate moiety containing triglycerides in the nutritional composition.

In one embodiment the compound having formula (1) comprises at least 30% by weight, preferably at least 40% by weight, of the total butyrate moiety containing triglycerides in the composition.

In a preferred embodiment the compound having formula (1) comprises at least 50% by weight of the total butyrate moiety containing triglycerides in the composition.

In another preferred embodiment the compound having formula (1) comprises at least 60% by weight of the total butyrate moiety containing triglycerides in the composition.

In one embodiment the compound having formula (1) comprises at least 70% by weight of the total butyrate moiety containing triglycerides in the composition.

In one embodiment the compound having formula (1) comprises at least 80% by weight of the total butyrate moiety containing triglycerides in the composition.

In one embodiment the compound having formula (1) comprises at least 90% by weight of the total butyrate moiety containing triglycerides in the composition.

In one embodiment the compound having formula (1) comprises at least 95% by weight of the total butyrate moiety containing triglycerides in the composition.

According to an aspect of the present invention there is provided a nutritional composition for providing a source of butyrate or butyric acid, comprising a compound having the formula

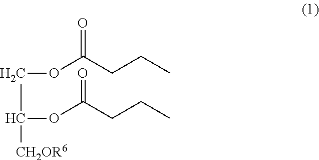

wherein R⁶ is a long chain fatty acid having between 16 and 20 carbons, and optionally a compound of formula

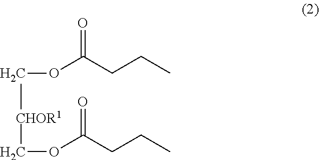

wherein R¹ is a long chain fatty acid having between 16 and 20 carbons, wherein the compound of formula (1), optionally in combination with the compound of formula (2), are present in an amount of at least 30%, preferably at least 40%, more preferably at least 50% by weight of the total butyrate moiety containing triglycerides in the composition.

In one embodiment a compound having formula (1) and a compound having the formula (2) are present in the composition (e.g., nutritional composition, dietary supplement, infant formula or follow on formula), as defined herein, wherein the combination of the compound having formula (1) and the compound having the formula (2) is present in an amount of at least 50% by weight, preferably at least 60% by weight of the total butyrate moiety containing triglycerides in the composition.

In another embodiment a compound having formula (1) and a compound having the formula (2) are present in the composition (e.g., nutritional composition, dietary supplement, infant formula or follow on formula), as defined herein, wherein the combination of the compound having formula (1) and the compound having the formula (2) is present in an amount of at least 60%, at least 70%, at least 80% or at least 90% by weight of the total butyrate moiety containing triglycerides in the composition.

In an embodiment the nutritional composition comprises less than 15% by weight, preferably less than 10% by weight of the total butyrate moiety containing triglycerides in the composition, of a compound having the formula

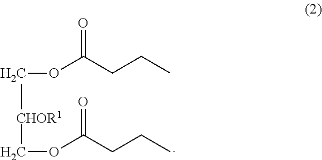

less than 10% by weight of the total butyrate moiety containing triglycerides in the composition, of a compound having the formula

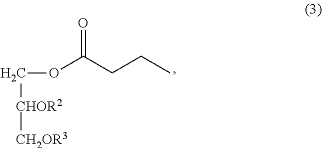

and/or,

less than 10% by weight of the total butyrate moiety containing triglycerides in the composition, of a compound having the formula

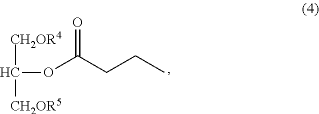

wherein R¹, R², R³, R⁴ and R⁵ are each independently a long chain fatty acid having between 16 and 20 carbons.

In an embodiment the nutritional composition comprises less than 8% by weight, preferably less than 5% weight, or less than 2% by weight of the total butyrate moiety containing triglycerides in the composition, of a compound having the formula (2).

In a preferred embodiment the nutritional composition comprises less than 15% by weight, of the total butyrate moiety containing triglycerides in the composition, of a combination of the compound having the formula (3) and the compound having the formula (4).

In an embodiment the nutritional composition comprises less than 8% by weight, preferably less than 5% weight, or less than 2% by weight of the total butyrate moiety containing triglycerides in the composition, of a compound having the formula (3).

In an embodiment the nutritional composition comprises less than 8% by weight, preferably less than 5% weight, or less than 2% by weight of the total butyrate moiety containing triglycerides in the composition, of a compound having the formula (4).

In an embodiment the nutritional composition may be selected from a dietary supplement, a fortified food, a medical food or food for special medical purposes, an infant formula, a follow-on formula.

According to one aspect of the present invention there is provided a dietary supplement for providing a source of butyrate or butyric acid comprising a compound having the formula

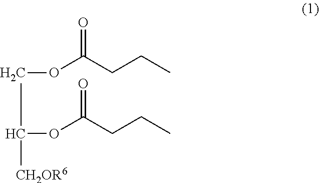

wherein R⁶ is a long chain fatty acid having between 16 and 20 carbons, as the main source of the butyrate or butyric acid in the dietary supplement.

The dietary supplement may be in the form of, for example, a capsule, tablet, sachet, a liquid/oil or powder.

According to another aspect of the present invention there is provided an infant formula or follow-on formula comprising a compound having the formula

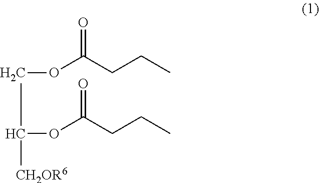

wherein R⁶ is independently, a long chain fatty acid having between 16 and 20 carbons, as a main source of the butyrate or butyric acid in the infant formula or follow-on formula.

According to another aspect of the present invention there is provided use of the infant formula or follow-on formula of the present invention for providing a source of butyrate or butyric acid with improved organoleptic properties.

According to another aspect of the present invention there is provided a nutritional composition comprising a compound having the formula

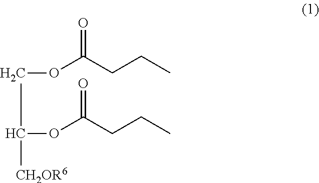

wherein R⁶ is a long chain fatty acid having between 16 and 20 carbons, as the main source of butyrate or butyric acid in the nutritional composition, for use in improving or maintaining gastrointestinal (GI) health.

According to another aspect of the present invention there is provided a method of improving or maintaining GI health in a patient comprising administering to said patient an effective amount of a nutritional composition comprising a compound having the formula

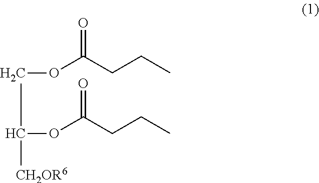

wherein R⁶ is a long chain fatty acid having between 16 and 20 carbons, as the main source of butyrate or butyric acid in the nutritional composition.

In a preferred embodiment the compound of formula (1) is the main butyrate moiety containing triglyceride component in the nutritional composition.

In one embodiment, a compound having formula (1) is used as defined herein, or is present in the composition (e.g., nutritional composition, dietary supplement, infant formula or follow on formula) as defined herein. Preferably the compound having formula (1) is present in an amount of at least 2% by weight, preferably at least 3% by weight, such as about 3 to 6% by weight of the total triglycerides in the composition.

In one embodiment, a compound having formula (1) is used as defined herein, or is present in a dietary supplement, as defined herein, in an amount of at least 5% by weight, preferably at least 10% by weight, such as at least 15% or at least 20% by weight of the total triglycerides in the composition.

In one embodiment a compound having formula (1) is used as defined herein, or is present in the composition (e.g., nutritional composition, dietary supplement, infant formula or follow-on formula) as defined herein, wherein the compound having formula (1) is present in an amount of at least 20% by weight of the total butyrate moiety containing triglycerides in the composition.

In a preferred embodiment a compound having formula (1) is used as defined herein, or is present in the composition (e.g., nutritional composition, dietary supplement, infant formula or follow on formula) as defined herein wherein the compound having formula (1) is present in an amount of at least 30% by weight, preferably at least 40% by weight, or preferably at least 50% by weight of the total butyrate moiety containing triglycerides in the composition.

In another embodiment a compound having formula (1) is used as defined herein, or is present in the composition (e.g., nutritional composition, dietary supplement, infant formula or follow on formula) as defined herein wherein the compound having formula (1) is present in an amount of at least 60%, at least 70%, at least 80% or at least 90% by weight of the total butyrate moiety containing triglycerides in the composition.

In another embodiment a compound having formula (1) is used as defined herein, or is present in the composition (e.g., nutritional composition, dietary supplement, infant formula or follow on formula) as defined herein wherein the compound having formula (1) is present in an amount of at least 95% by weight of the total butyrate moiety containing triglycerides in the composition.

In one embodiment, R⁶ as defined herein is an unsaturated fatty acid, preferably monounsaturated.

In one embodiment, R⁶ as defined herein is selected from the group consisting of oleic acid, palmitic acid, stearic acid or linoleic acid.

In a preferred embodiment, R⁶ as defined herein is oleic acid.

In one embodiment, the compound having the formula (1) is:

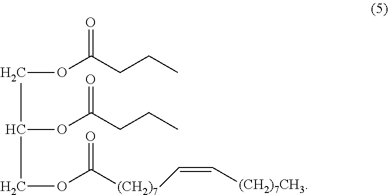

According to another aspect of the present invention there is provided a composition comprising the compound having the formula

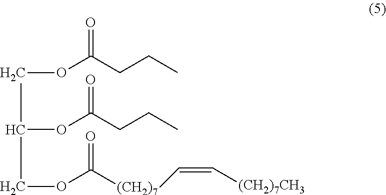

wherein the compound having formula (5) comprises at least 2% by weight, at least 3% by weight, at least 5% by weight or at least 10% by weight of the total triglycerides in the composition.

In one embodiment the compound having formula (5) comprises at least 20% by weight of the total butyrate moiety containing triglycerides in the composition.

In one embodiment the compound having formula (5) comprises at least 30% by weight of the total butyrate moiety containing triglycerides in the composition.

In one embodiment the compound having formula (5) comprises at least 40% by weight of the total butyrate moiety containing triglycerides in the composition.

In one embodiment the compound having formula (5) comprises at least 50% by weight of the total butyrate moiety containing triglycerides in the composition.

In one embodiment the compound having formula (5) comprises at least 60% by weight of the total butyrate moiety containing triglycerides in the composition.

In one embodiment the compound having formula (5) comprises at least 70% by weight of the total butyrate moiety containing triglycerides in the composition.

In one embodiment the compound having formula (5) comprises at least 80% by weight of the total butyrate moiety containing triglycerides in the composition.

In one embodiment the compound having formula (5) comprises at least 90% by weight of the total butyrate moiety containing triglycerides in the composition.

In one embodiment the compound having formula (5) comprises at least 95% by weight of the total butyrate moiety containing triglycerides in the composition.

In one embodiment the compound having formula (5) comprises about 20% to about 95% by weight of the total butyrate moiety containing triglycerides in the composition, about 30% to about 95%, about 40% to about 90%, about 50% to about 90%, or about 60% to about 95% by weight of the total butyrate moiety containing triglycerides in the composition.

In one embodiment the compound having formula (5) comprises about 40% to about 80% by weight of the total butyrate moiety containing triglycerides in the composition, for example about 50% to about 70% by weight of the total butyrate moiety containing triglycerides in the composition.

In one embodiment the compound having formula (5) comprises about 50% to about 90% by weight of the total butyrate moiety containing triglycerides in the composition, for example about 60% to about 80% by weight of the total butyrate moiety containing triglycerides in the composition.

According to an aspect of the present invention there is provided a nutritional composition for providing a source of butyrate or butyric acid, comprising a compound having the formula

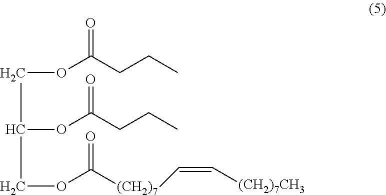

and, optionally a compound of formula

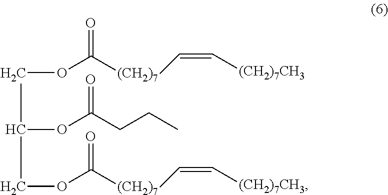

wherein the compound of formula (5), optionally in combination with the compound of formula (6), are present in an amount of at least 30%, preferably at least 40%, more preferably at least 50% by weight of the total butyrate moiety containing triglycerides in the composition.

In one embodiment a compound having formula (5) and a compound having the formula (6) are present in the composition (e.g., nutritional composition, dietary supplement, infant formula or follow on formula), as defined herein, wherein the combination of the compound having formula (5) and the compound having the formula (6) is present in an amount of at least 50% by weight, preferably at least 60% by weight of the total butyrate moiety containing triglycerides in the composition.

In another embodiment a compound having formula (5) and a compound having the formula (6) are present in the composition (e.g., nutritional composition, dietary supplement, infant formula or follow on formula), as defined herein, wherein the combination of the compound having formula (5) and the compound having the formula (6) is present in an amount of at least 60%, at least 70%, at least 80% or at least 90% by weight of the total butyrate moiety containing triglycerides in the composition.

In one embodiment the composition comprises less than 10% by weight of the total butyrate moiety containing triglycerides in the composition of a compound having the formula

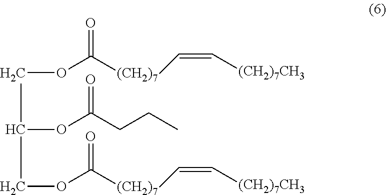

According to an embodiment the composition comprises less than 10% by weight of the total butyrate moiety containing triglycerides in the composition, of a compound having the formula

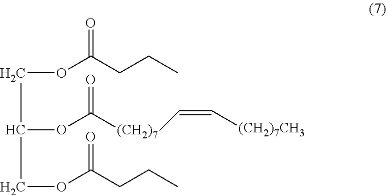

According to an embodiment the composition comprises less than 10% by weight of the total butyrate moiety containing triglycerides in the composition, of a compound having the formula

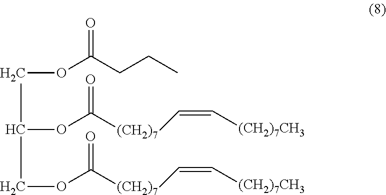

In an embodiment the nutritional composition comprises less than 8% by weight, preferably less than 5% weight, or less than 2% by weight of the total butyrate moiety containing triglycerides in the composition, of a compound having the formula (6).

In a preferred embodiment the nutritional composition comprises less than 15% by weight, of the total butyrate moiety containing triglycerides in the composition, of a combination of the compound having the formula (7) and the compound having the formula (8).

In an embodiment the nutritional composition comprises less than 8% by weight, preferably less than 5% weight, or less than 2% by weight of the total butyrate moiety containing triglycerides in the composition, of a compound having the formula (7).

In an embodiment the nutritional composition comprises less than 8% by weight, preferably less than 5% weight, or less than 2% by weight of the total butyrate moiety containing triglycerides in the composition, of a compound having the formula (8).

The composition of the present invention may further comprise 1,3-dibutyryl-2-linoleoylglycerol, 1,3-dibutyryl-2-stearoylglycerol, 1-butyryl-2-oleoyl-3-palmitoylglycerol, 1-palmitoyl-2-oleoyl-3-butyrylglycerol, 1-butyryl-2-oleoyl-3-linoleoylglycerol, 1-linoleoyl-2-oleoyl-3-butyrylglycerol, 1-oleoyl-2-butyryl-3-linoleoylglycerol, 1-linoleoyl-2-butyryl-3-oleoylglycerol, 1-butyryl-2-linoleoyl-3-oleoylglycerol, 1-oleoyl-2-linoleoyl-3-butyrylglycerol, 1-butyryl-2-stearoyl-3-oleoylglycerol, 1-oleoyl-2-stearoyl-3-butyrylglycerol, 1-butyryl-2-oleoyl-3-stearoylglycerol, 1-stearoyl-2-oleoyl-3-butyrylglycerol, 1,2-dioleoyl-3-palmitoylglycerol, 1-palmitoyl-2,3-dioleoylglycerol, 1,2-dioleoyl-3-linoleoylglycerol and/or 1-linoleoyl-2,3-dioleoylglycerol.

The composition of the present invention is preferably in the form of nutritional composition.

The composition of the present invention may be in the form of an infant formula or follow on formula.

The composition of the present invention may be in the form of dietary supplement.

According to another aspect of the present invention there is provided use of a composition defined herein for providing a source of butyrate or butyric acid with improved organoleptic properties.

According to another aspect of the present invention there is provided a method of providing a source of butyric acid with improved organoleptic properties to a subject, said method comprising administering an effective amount of a composition defined herein to said subject.

According to another aspect of the present invention there is provided a composition defined herein for improving or maintaining gastrointestinal health.

According to another aspect of the present invention there is provided a method of improving or maintaining gastrointestinal health in a subject comprising administering an effective amount of a composition defined herein to a subject.

### DETAILED DESCRIPTION OF THE INVENTION

Triglycerides

A triglyceride (also known as a triacylglycerol) is a triester that is derived from glycerol and three fatty acids.

Fatty acids are carboxylic acids with a long tail (chain). Fatty acids may be either unsaturated or saturated. Fatty acids which are not attached to other molecules are referred to as free fatty acids (FFA).

The term “fatty acid moiety” refers to the part of the triglyceride that originates from a fatty acid in an esterification reaction with glycerol. The triglycerides used in the present invention comprise two butyric acid moieties (also referred to as “butyrate moieties”) and one long chain fatty acid moiety.

Advantageously compounds and compositions of the present invention can provide high butyric acid content, through optimization of the butyric acid content of the triglyceride, whilst exhibiting improved organoleptic properties compared to tributyrin.

Preferred long chain fatty acids for use in the present invention are fatty acids that have 16 to 20 carbon atoms.

Examples of long chain fatty acid include oleic acid, palmitic acid, stearic acid and linoleic acid.

A preferred long chain fatty acid for use in the present invention is oleic acid. Oleic acid is the predominant fatty acid present in breast milk.

The triglycerides of the present invention may be synthesised by, for example, esterification of a long chain fatty acid monoacylglycerol (MAG) with butyric acid.

The triglycerides of the present invention may be synthesised by, for example, esterification of long chain fatty acid monoacylglycerol (MAG) with butyric acid (BA) with the removal of water. By way of example a method of obtaining 1,2-dibutyryl-3-oleoylglycerol is shown below:

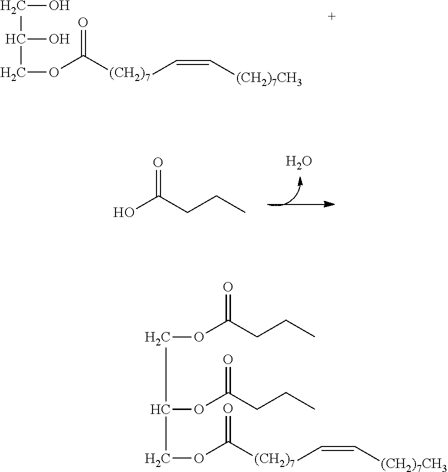

The esterification reaction is preferably carried out with butyric acid (BA): monoacylglycerol (MAG) molar ratio of 2; i.e. in a molar excess of butyric acid, for example a molar ratio BA:MAG of about 3.

Removal of water can be carried by conventional methods, routinely determined by a person skilled in the art.

Advantageously, such methods of synthesis avoid the use of tributyrin as a raw material.

Alternative methods of triglyceride synthesis can be routinely determined by a person skilled in the art.

In one embodiment, sunflower oil is the source of monoolein as monoacylglycerol. This generates triglycerides containing predominantly butyrate and oleate moieties.

The compounds are dairy-free, cholesterol-free and vegan. Fatty acids are liberated from triglycerides due to lipases, naturally present in the gastrointestinal tract. Relative to butyrate salts, the compounds do not add additional mineral salts to the final formulation.

A single butyrate moiety containing triglyceride may be used herein. Alternatively, a mixture of different butyrate moiety containing triglycerides may be used.

The triglycerides may be further subjected to decoloration and/or deodorization steps conventional in the art and well-known to the person skilled in the art. For example, as conventionally used in the manufacture of vegetable oils.

Compositions

The present invention provides compositions comprising butyrate moiety containing triglycerides referred to herein. The composition may be, for example, a nutritional composition, including a fortified food, a medical food or food for special medical purposes, a dietary supplement, an infant formula or a follow-on formula.

The expression “nutritional composition” means a composition that nourishes a subject. This nutritional composition is preferably taken orally, and it may include a lipid or fat source and a protein source. It may also contain a carbohydrate source. In one embodiment, the nutritional composition contains only a lipid or fat source. In other specific embodiments, the nutritional composition contains a lipid (or fat) source with a protein source, a carbohydrate source or both.

In some specific embodiments, the nutritional composition according to the invention is a food for special dietary uses (FSDU). The expression “food for special dietary uses” as used herein refers to a foodstuff which is specially formulated to satisfy particular dietary requirements which exist because of a particular physical or physiological condition and/or specific diseases and disorders (e.g. CODEX STAN 146-1985, section 2.1)

In some specific embodiments, the nutritional composition according to the invention is a medical food or a food for special medial purposes (FSMP). “Medical foods” or food for special medical purposes” as used herein refers to foods that are specially formulated and intended for the dietary management of a disease. In the United States “Medical Foods” were defined in section 5(b) of the Food and Drug Administration's 1988 Orphan Drug Act (21 U.S.C. 360ee (b) (3)). In Europe “Medical Foods” are referred to a “Foods for special medical purposes” (e.g. as defined under European Commission Delegated Regulation (EU) 2016/128 of 25 Sep. 2015). The terms “medical food” and “foods for special medical purposes” are used interchangeably herein.

In some specific embodiments, the nutritional composition according to the invention is an “enteral nutritional composition” that is to say a foodstuff that involves the gastrointestinal tract for its administration. The gastric introduction may involve the use of a tube through the oro/nasal passage or a tube in the belly leading directly to the stomach. This may be used especially in hospitals or clinics.

The composition according to the invention can be an infant formula (e.g. a starter infant formula), a follow-up or follow-on formula, a growing-up milk, a baby food, an infant cereal composition, a fortifier such as a human milk fortifier, or a supplement.

The expression “infant formula” as used herein refers to a foodstuff intended for particular nutritional use by infants during the first months of life and satisfying by itself the nutritional requirements of this category of person (e.g., Article 2(c) of the European Commission Directive 91/321/EEC 2006/141/EC of 22 Dec. 2006 on infant formulae and follow-on formulae).

Generally a starter formula is for infants from birth as breast-milk substitute. A follow-up or follow-on formula is given from the sixth month onwards. It constitutes the principal liquid element in the progressively diversified diet of this category of person. The “growing-up milks” (or GUMs) are given from one year onwards. It is generally a milk-based beverage adapted for the specific nutritional needs of young children.

The term “fortifier” refers to liquid or solid nutritional compositions suitable for mixing with breast milk (human milk) or infant formula. The term “breast milk” should be understood as the mother's milk or the colostrum of the mother or a donor's milk or the colostrum of a donor's milk.

The term “dietary supplement” may be used to complement the nutrition of an individual (it is typically used as such but it might also be added to any kind of compositions intended to be ingested). It may be in the form of tablets, capsules, pastilles or a liquid for example. The supplement may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents and gel forming agents. The dietary supplement may also contain conventional pharmaceutical additives and adjuvants, excipients and diluents, including, but not limited to, water, gelatine of any origin, vegetable gums, lignin-sulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like.

In another particular embodiment the nutritional composition of the present invention is a fortifier. The fortifier can be a breast milk fortifier or a formula fortifier such as an infant formula fortifier. The fortifier is therefore a particularly advantageous embodiment when the infant or young child is born preterm.

When the composition is a supplement, it can be provided in the form of unit doses.

The nutritional composition of the invention, and especially the infant formula, generally contains a protein source, a carbohydrate source and a lipid source. In some embodiments however, especially if the nutritional composition of the invention is a supplement or a fortifier, there may be only lipids (or a lipid source).

The nutritional composition according to the invention may contain a protein source. The protein may be in an amount of from 1.6 to 3 g per 100 kcal. In some embodiments, especially when the composition is intended for preterm infants/young children, the protein amount can be between 2.4 and 4 g/100 kcal or more than 3.6 g/100 kcal. In some other embodiments the protein amount can be below 2.0 g per 100 kcal, e.g. between 1.8 to 2 g/100 kcal, or in an amount below 1.8 g per 100 kcal.

Protein sources based on, for example, whey, casein and mixtures thereof may be used as well as plant-based protein sources, for example, based on soy. As far as whey proteins are concerned, the protein source may be based on acid whey or sweet whey or mixtures thereof and may include alpha-lactalbumin and beta-lactoglobulin in any desired proportions. In some embodiments the protein source is whey predominant (i.e. more than 50% of proteins are coming from whey proteins, such as 60%> or 70%>). The proteins may be intact or hydrolysed or a mixture of intact and hydrolysed proteins. By the term “intact” is meant that the main part of the proteins are intact, i.e. the molecular structure is not altered, for example at least 80% of the proteins are not altered, such as at least 85% of the proteins are not altered, preferably at least 90% of the proteins are not altered, even more preferably at least 95% of the proteins are not altered, such as at least 98% of the proteins are not altered. In a particular embodiment, 100% of the proteins are not altered.

The term “hydrolysed” means in the context of the present invention a protein which has been hydrolysed or broken down into its component amino acids.

The proteins may be either fully or partially hydrolysed. If hydrolysed proteins are required, the hydrolysis process may be carried out as desired and as is known in the art. For example, whey protein hydrolysates may be prepared by enzymatically hydrolysing the whey fraction in one or more steps. If the whey fraction used as the starting material is substantially lactose free, it is found that the protein suffers much less lysine blockage during the hydrolysis process. This enables the extent of lysine blockage to be reduced from about 15% by weight of total lysine to less than about 10%> by weight of lysine; for example about 7% by weight of lysine which greatly improves the nutritional quality of the protein source.

In one particular embodiment the proteins of the composition are hydrolysed, fully hydrolysed or partially hydrolysed. The degree of hydrolysis (DH) of the protein can be between 2 and 20, or between 8 and 40, or between 20 and 60 or between 20 and 80 or more than 10, 20, 40, 60, 80 or 90. For example, nutritional compositions containing hydrolysates having a degree of hydrolysis less than about 15% are commercially available from Nestle Company under the trade mark Peptamen®.

At least 70%, 80%, 85%, 90%, 95% or 97% of the proteins may be hydrolysed. In a particular embodiment, 100% of the proteins are hydrolysed.

In one particular embodiment the proteins of the composition are plant-based protein.

The nutritional composition according to the present invention may contain a carbohydrate source. This is particularly preferable in the case where the nutritional composition of the invention is an infant formula. In this case, any carbohydrate source conventionally found in infant formulae such as lactose, sucrose, saccharose, maltodextrin, starch and mixtures thereof may be used although one of the preferred sources of carbohydrates for infant formula is lactose. The nutritional composition of the invention may also contain all vitamins and minerals understood to be essential in the daily diet and in nutritionally significant amounts. Minimum requirements have been established for certain vitamins and minerals. Examples of minerals, vitamins and other nutrients optionally present in the composition of the invention include vitamin A, vitamin B1, vitamin B2, vitamin B3, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chlorine, potassium, sodium, selenium, chromium, molybdenum, taurine, and L-carnitine. Minerals are usually added in salt form. The presence and amounts of specific minerals and other vitamins will vary depending on the intended population. If necessary, the nutritional composition of the invention may contain emulsifiers and stabilisers such as soy, lecithin, citric acid esters of mono- and diglycerides, and the like. The nutritional composition of the invention may also contain other substances which may have a beneficial effect such as lactoferrin, osteopontin, TGFbeta, slgA, glutamine, nucleotides, nucleosides, and the like.

The composition of the invention can further comprise at least one non-digestible oligosaccharide (e.g. prebiotics). They are usually in an amount between 0.3 and 10% by weight of composition.

Prebiotics are usually non-digestible in the sense that they are not broken down and absorbed in the stomach or small intestine and thus remain intact when they pass into the colon where they are selectively fermented by the beneficial bacteria. Examples of prebiotics include certain oligosaccharides, such as fructooligosaccharides (FOS), inulin, xylooligosaccharides (XOS), polydextrose or any mixture thereof. In a particular embodiment, the prebiotics may be fructooligosaccharides and/or inulin. In a specific embodiment, the prebiotics is a combination of FOS with inulin such as in the product sold by BENEO-Orafti under the trademark Orafti® oligofructose (previously Raftilose®) or in the product sold by BENEO-Orafti under the trademark Orafti® inulin (previously Raftiline®). Another example is a combination of 70% short chain fructooligosaccharides and 30% inulin, which is registered by Nestle under the trademark “Prebio 1”. The nutritional composition of the invention can also comprise at least one milk oligosaccharide that can be a BMO (bovine milk oligosaccharide) and/or a HMO (human milk oligosaccharide). The composition of the present invention can further comprise at least one probiotic (or probiotic strain), such as a probiotic bacterial strain.

The probiotic microorganisms most commonly used are principally bacteria and yeasts of the following genera: Lactobacillus spp., Streptococcus spp., Enterococcus spp., Bifidobacterium spp. and Saccharomyces spp.

In some particular embodiments, the probiotic is a probiotic bacterial strain. In some specific embodiments, it is Bifidobacteria and/or Lactobacilli.

The nutritional composition according to the invention may contain from 10e3 to 10e12 cfu of probiotic strain, more preferably between 10e7 and 10e12 cfu such as between 10e8 and 10e10 cfu of probiotic strain per g of composition on a dry weight basis.

In one embodiment the probiotics are viable. In another embodiment the probiotics are non-replicating or inactivated. It may also be probiotic parts such as cell wall components or products of the probiotic metabolism. There may be both viable probiotics and inactivated probiotics in some other embodiments. The nutritional composition of the invention can further comprise at least one phage (bacteriophage) or a mixture of phages, preferably directed against pathogenic Streptococci, Haemophilus, Moraxella and Staphylococci.

The nutritional composition according to the present invention can in one embodiment be a dairy product. Dairy products are products comprising milk-based products. Dairy products are generally made from a suitable mixture of concentrated milk protein and fat sources. Dairy products may be acidified. Dairy products include ready-to-drink milk-based beverages, concentrated milk, evaporated milk, sweetened and condensed milk, milk powder, yoghurts, fresh cheese, cheese, ice-cream and dairy spreads such as spreadable fresh cheese, cottage cheese, quark, crème fraiche, clotted cream and cream cheese. Milk powder may be manufactured, for instance, by spray-drying or by freeze-drying.

Depending on their fat content, dairy products may be prepared from full-fat or whole milk, semi-skim milk, skim or low-fat milk. Skim milk is a milk that contains less than 0.1% milk fat. Semi-skim milk is milk that contains between 1.5% and 2.5% milk fat. Usually, full-fat milk is milk that contains 3% to 4% fat. The exact fat content for skim, semi-skim and full-fat milks depend mainly on local food regulation.

Dairy products are generally made from cow milk. Dairy products may also be made from buffalo milk, yak milk, goat milk, ewe milk, mare milk, donkey milk, camel milk, reindeer milk, moose milk, or combinations thereof.

Acidified dairy products may be obtained by fermentation with suitable micro-organisms. Fermentation provides flavour and acidity to the dairy product. It may also affect the texture of the dairy product. In addition, micro-organisms employed in fermentation are selected for their capacity to ferment milk into a consumable fermented dairy product. Usually, said microorganisms are known for their beneficial properties. Said micro-organisms include lactic acid bacteria and yeasts. Some of these micro-organisms may be considered as probiotics. Examples of lactic acid bacteria include Lactobacillus delbrueckii subsp. bulgaricus and Streptococcus thermophilus, both of which are involved in production of yogurt, or other lactic acid bacteria belonging to the genera Lactobacillus, Streptococcus, Lactococcus, Leuconostoc, Bifidobacterium, Pediococcus or any mixture thereof.

Another example of fermented dairy products, also known as cultured dairy products or cultured dairy foods, or cultured milks, is cultured buttermilk fermented with Lactococcus lactis (Lactococcus lactis subsp. lactis, Lactococcus lactis subsp. cremoris, Lactococcus lactis subsp. lactis biovar. diacetylactis) and/or Leuconostoc mesenteroides subsp. cremoris.

The micro-organisms may be live or inactivated.

Dairy product analogues are products made in a similar way to the dairy products above, but where (fully or partly) a non-milk source of protein is used and/or (fully or partly) a non-dairy source of edible fat. Suitable protein sources include vegetable proteins such as soy, potato and pea. Suitable fat sources include oils and fats from vegetable or marine origin. Fats and oils are used as interchangeable terms. Similar preparation as referred to above are meant to include processes for products in which a traditional whey separation step is omitted because the formulation of the dairy analogue of the product allows skipping this step.

The nutritional composition according to the invention may be prepared in any suitable manner.

For example, a formula such as an infant formula may be prepared by blending together the protein source, the carbohydrate source and the fat source, in appropriate proportions. If used, the emulsifiers may be included at this point. The vitamins and minerals may be added at this point but they are usually added later to avoid thermal degradation. Any lipophilic vitamins, emulsifiers and the like may be dissolved into the fat source prior to blending. Water, preferably water that has been subjected to reverse osmosis, may then be mixed in to form a liquid mixture. The temperature of the water is conveniently in the range between about 50° C. and about 80° C. to aid dispersal of the ingredients. Commercially available liquefiers may be used to form the liquid mixture.

Any oligosaccharides may be added at this stage, especially if the final product is to have a liquid form. If the final product is to be a powder, they may likewise be added at this stage if desired.

The liquid mixture is then homogenised, for example in two stages.

In one embodiment the nutritional composition of the invention is given to the infant or young child as a supplementary composition to the mother's milk.

The composition of the present invention can be in, for example, a solid (e.g. powder), liquid or gelatinous form.

The composition of the present invention can be in, for example, tablet, dragee, capsule, gel cap, powder, granule, solution, emulsion, suspension, coated particle, spray-dried particle or pill.

The composition may in the form of a pharmaceutical composition and may comprise one or more suitable pharmaceutically acceptable carriers, diluents and/or excipients.

Examples of such suitable excipients for compositions described herein may be found in the “Handbook of Pharmaceutical Excipients”, 2nd Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

The pharmaceutical compositions may comprise as, or in addition to, the carrier, excipient or diluent any suitable binder(s), lubricant(s), suspending agent(s), coating agent(s) and/or solubilising agent(s). Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilisers, dyes and even flavouring agents may be provided in the composition. Examples of preservatives include sodium benzoate, sorbic acid and esters of p-hydroxybenzoic acid. Antioxidants and suspending agents may be also used.

Gastrointestinal Health

The compounds defined herein are a source of butyrate/butyric acid and may therefore be used for improving or maintaining gastrointestinal (GI) health.

In one embodiment, the compounds and compositions defined herein may be used for treating inflammatory bowel disease, for example, Crohn's Disease or Ulcerative Colitis.

The multiple beneficial effects on GI health of butyrate are well documented. At the intestinal level, butyrate plays a regulatory role on the transepithelial fluid transport, mucosal inflammation and oxidative status, reinforces the epithelial barrier, and modulates visceral sensitivity and intestinal motility.

Fatty acids, including butyric acid, are a chief source of energy for cells of the colonic mucosa (Roedriger, Gut. 1980; 21: 793-798), and of the greatest importance to colonocytes in the distal region of the colon. The strong trophic effect of butyric acid on the mucous membrane of the small intestine has been observed in experimental animals (Guilloteau et al., 2 J Anim Feed Sci. 2004; 13, Suppl. 1: 393-396). A decrease in the intestinal butyric acid concentration leads to the atrophy of colonic mucosa, which is usually explained by a decrease in the availability of substrates to colonocytes. On the other hand, administration of butyrate into the colon lumen induces weight gain, an increase in DNA synthesis and the depth of intestinal crypts (Kripke et al., J Parenter Enter Nutr. 1989; 13: 109-116).

A high concentration of butyric acid achieved through the fermentation of insoluble dietary fiber or following the anal administration of butyrate may inhibit early and late stages of colon oncogenesis through the regulation of transcription, expression and activation of key proteins of the apoptotic cascade (Avivi-Green et al., J Nutr. 2002; 132 (7): 1812-18).

Chapman et al. (Gut 1994; 35(1): 73-76) showed that inflamed colonic mucosa captures much more butyrate than glutamine or glucose. Experiments have shown that butyrate perfusions result in a significant reduction of inflammation, and a decrease in the extent of ulceration of the colon wall in the rat (Andoh et al., J Parenter Enter Nutr. 1999; 23(5): 70-73).

The effectiveness of butyrate enemas has been shown by clinical observations in patients with Ulcerative Colitis (Han et al., Gastroenterol Clin North Am. 1999; 28: 423-443; Scheppach et al., Gastroenterol Suppl. 1997; 222: 53-57).

The direct anti-inflammatory activity of butyrate may be connected with the inhibition of the migration of the KappaB nuclear factor (NFKB) and its binding of DNA, and by the same token the inhibition of transcription and production of pro-inflammatory cytokines (Segain et al., Gut. 2000; 47: 397-403).

Accordingly, the triglyceride compounds used in the present invention, which are a source of butyrate, can provide a significant role in the maintenance of intestinal homeostasis and GI health.

Administration

Preferably, the compounds and compositions described herein are administered enterally.

Enteral administration may be for example oral or gastric.

In general terms, administration of the combination or composition described herein may, for example, be by an oral route or another route into the gastro-intestinal tract, for example the administration may be by tube feeding.

The subject may be a mammal such as a human, canine, feline, equine, caprine, bovine, ovine, porcine, cervine and primates. Preferably the subject is a human.

### EXAMPLES

### Example 1—Preparation of Butyrate Moiety Containing Triglycerides

Compositions comprising butyrate moiety containing triglycerides were generated by the esterification reaction between monoolein (derived from sunflower oil) with butyric acid added in molar excess (5 equivalents in total). These two reagents were mixed together in a flask and heated to reflux (butyric acid boiling point is 163.5° C.). A condenser (“colonne de Vigreux”) was used to remove the water. The reaction was monitored by TLC and stopped when all the monoacylglycerol was converted into triacylglycerol.

The constituents, mostly triglycerides, of the resulting oil compositions are shown below in Table 1. These triglycerides are represented by the three fatty acids they contain. These fatty acids are represented by their lipid number: 4:0 for butyrate, 16:0 for palmitate, 18:0 for stearate 18:1 for oleate and 18:2 for linoleate. The fatty acid in the middle is located on the position sn-2 in the triglyceride. As an example, 16:0-4:0-18:1 stands for two different triglycerides having both a butyrate in position sn-2 and either a palmitate in position sn-1 and an oleate in position sn-3 or an oleate in position sn-1 and a palmitate in position sn-3.

TABLE 1
Trigylceride profile [% by weight]

| | |
|---|---|
| 4:0-4:0-18:1&4:0-18:1-4:0 | 65.64 |
| 18:1-18:1-4:0&18:1-4:0-18:1 | 12.53 |
| 4:0-4:0-18:2&4:0-18:2-4:0 | 5.43 |
| 4:0-4:0-18:0&4:0-18:0-4:0 | 3.03 |
| 4:0-18:1-18:2&isomers | 2.98 |
| 4:0-16:0-18:1&isomers | 1.69 |
| 4:0-4:0-16:0&4:0-16:0-4:0 | 1.40 |
| 4:0-4:0-4:0 | 1.36 |
| 4:0-18:0-18:1&isomers | 0.99 |
| 4:0-4:0-22:0&4:0-22:0-4:0 | 0.82 |
| 18:1-18:1-18:1 | 0.63 |
| 4:0-22:0-18:1&isomers | 0.33 |
| 4:0-4:0-24:0&4:0-24:0-4:0 | 0.31 |
| 4:0-4:0-20:0&4:0-20:0-4:0 | 0.28 |
| 4:0-16:0-18:0&isomers | 0.25 |
| 18:0-18:0-16:0 | 0.22 |
| 4:0-16:0-18:2&isomers | 0.21 |
| 4:0-4:0-20:1&4:0-20:1-4:0 | 0.20 |
| 18:1-18:1-18:2 | 0.17 |
| 18:2-18:2-4:0&18:2-4:0-18:2 | 0.17 |
| 18:0-18:0-4:0&18:0-4:0-18:0 | 0.16 |
| 16:0-16:0-4:0&16:0-4:0-16:0 | 0.14 |
| 16:0-18:0-16:0 | 0.12 |
| 4:0-4:0-18:3&4:0-18:3-4:0 | 0.11 |
| 4:0-4:0-16:1&4:0-16:1-4:0 | 0.11 |

In the composition, 4:0-4:0-18:1 was identified as the most abundant triglyceride.

The resulting oil product was then subjected to a decoloration step with the action of bleaching earth and was purified either by short-path distillation (130° C., 0.001-0.003 mbar) and/or by deodorisation (160° C., 2 mbar, 2 h) with injection of steam water, to remove residual reagents and intermediates e.g. butyric acid, MAG and byproducts e.g. DAG and tributyrin.

The resulting oil product was evaluated in a descriptive sensory evaluation and found to have a better odour and taste than tributyrin and butyric acid.

1. A method for providing a source of butyrate or butyric acid comprising administering to a subject in need of same a compound having the formula 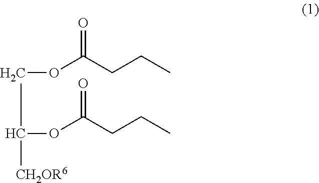
wherein R⁶ is a long chain fatty acid having between 16 and 20 carbons, as the main butyrate moiety containing triglyceride in the nutritional composition.

2. A method according to claim 1, wherein the compound of formula (1) is the main source of the butyrate or butyric acid, in the nutritional composition.

3. A according to claim 1, wherein the compound of formula (1) provides at least 20% of the butyrate moiety containing triglyceride provided by the nutritional composition.

4. A method according to claim 1 wherein the nutritional composition is a dietary supplement for providing a source of butyrate.

5. A method according to claim 4 in the form of a capsule, liquid, tablet, sachet or powder.

6. A method according to claim 1, wherein the nutritional composition is in the form of an infant formula or follow-on formula.

7. A method according to claim 1, for use in improving or maintaining gastrointestinal (GI) health.

8. (canceled)

9. Method according to claim 1, wherein the composition is selected from the group consisting of a fortified food, a medical food or food for special medical purposes, a food for special dietary use, a dietary supplement, an infant formula or a follow on-formula.

10. A method according to claim 1, wherein the compound having formula (1) is present in a composition that comprises a compound having formula (1) in an amount of at least 3% by weight of the total triglycerides in the composition.

11. A method according to claim 1, wherein the compound having formula (1) is present in a composition that comprises a compound having formula (1) in an amount of at least 30% by weight of the total butyrate moiety containing triglycerides in the composition.

12. A method according to claim 1, wherein the compound having formula (1) is present in a composition that comprises the compound having formula (1) in an amount of at least 60% by weight.

13. A method according to claim 1, wherein R⁶ is selected from the group consisting of oleic acid, palmitic acid, and linoleic acid.

14. A nutritional composition comprising the compound having the formula 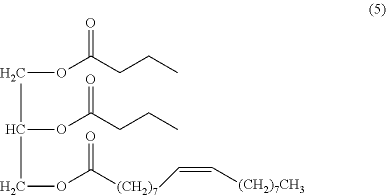
wherein the compound having formula (5) comprises at least 20% by weight of the total butyrate moiety containing triglycerides in the composition.

15. A nutritional composition according to claim 14 wherein the compound having the formula (5) comprises at least 3% by weight of the total triglycerides in the nutritional composition.

16. A nutritional composition according to claim 14 wherein the compound having formula (5) comprises at least 30% by weight of the total butyrate moiety containing triglycerides in the composition.

17. A nutritional composition according to claim 16 wherein the compound having formula (5) comprises at least 60% by weight of the total butyrate moiety containing triglycerides in the composition.

18. A composition according to claim 14 wherein the composition is an infant formula, a follow-on formula or a dietary supplement.

19-20. (canceled)
